(19) United States
(12) Patent Application Publication
(10) Pub. No.: US 2026/0318166 A1
(43) Pub. Date: Oct. 15, 2026

(54) BURROWING ROBOT AND BURROWING ROBOT HEAD

(71) Applicant: Regents of the University of Minnesota, Minneapolis, MN (US)

(72) Inventors: Nitish Ponkshe, Minneapolis, MN (US); James Van de Ven, Long Lake, MN (US)

(73) Assignee: Regents of the University of Minnesota, Minneapolis, MN (US)

(21) Appl. No.: 19/120,136

(22) PCT Filed: Oct. 12, 2023
(86) PCT No.: PCT/US2023/076664

§ 371 Date: Apr. 10, 2025

Related U.S. Application Data
(60) Provisional application No. 63/415,425, filed on Oct. 12, 2022.

(51) Int. Cl. E21B 23/00; E21B 7/04; E21B 10/26

(52) U.S. Cl. CPC E21B 23/001; E21B 7/04; E21B 10/26

(57) ABSTRACT

A burrowing robot head including a tip (or nose cone) and a plurality of expansion members configured to perform a two-step, penetration-expansion motion or process. The tip has a trailing end that defines a perimeter. The expansion members are arranged about the perimeter, and are each configured to transition between a retracted state and an expanded state. Each of the expansion members can include or comprise a linkage assembly, such as a 6-bar, two-dimensional linkage. The two-step, penetration-expansion process better ensures that the energy required to create a cavity in soil or other material is largely deformation energy and the frictional component of the energy is reduced significantly.

### GOVERNMENT SUPPORT CLAUSE

This invention was made with government support under EFRI-1830950 awarded by the National Science Foundation. The government has certain rights in the invention.

### BACKGROUND

The present disclosure relates to burrowing robots. More particularly, it relates to burrowing robot heads, and burrowing robots utilizing such heads, configured to form a cavity in soil or other material.

For centuries humans have explored the earth's subsurface in search of minerals and ground water reserves. Installation, inspection and maintenance of underground utilities like pipelines and cables need digging of soil to execute such operations. There have been instances of mine workers being trapped underground, with limited supply of fresh air and other essential commodities for survival, where it was imperative to rescue them in time. From a defense standpoint, locating and safely eliminating underground mines, and conducting stealth operations by digging underneath target locations requires significant subsurface navigation.

Conventional drilling operations, mostly requiring soil removal, have been used for a variety of the above applications. Recently, there has been a significant amount of research conducted in the area of underground robotics to tackle these, and other, problems. Such underground burrowing robots have the possibility to become effective alternatives to existing soil removal techniques and to provide solutions where conventional drilling techniques are less than optimal or unavailable. These robots can present the advantages of higher energy efficiency, compact size, more dexterity, ability to traverse tortuous paths, and faster speeds to reach target locations. Many such burrowing robots differ from conventional drilling techniques in their mode of cavity creation in that they rely on plastic deformation of soil by lateral compaction (with respect to the robot's direction of travel) instead of soil removal, as is the case with most, if not all, drilling techniques. Such an approach eliminates the need for having lines transporting cutting fluid to the drill point and transporting removed debris back to the surface.

While substantial efforts have been made in the design and construction of underground/burrowing robot bodies, minimal consideration has been given to the penetrating head of such robots. The head is responsible for initiating the cavity and allowing entry of the trailing robot body.

### SUMMARY

The inventors of the present disclosure have recognized that a need exists for a burrowing robot, and in particular a burrowing robot head, that overcomes one or more of the above-mentioned problems.

Some aspects of the present disclosure provide a burrowing robot head including a tip (or nose) and a plurality of expansion members. The tip has a trailing end that defines a perimeter. The expansion members are arranged about the perimeter, and are each configured to transition between a retracted state and an expanded state. In some embodiments, a footprint of the head in the expanded state is greater than a footprint in the retracted state. In some embodiments, each of the expansion members includes a linkage assembly, such as a two-dimensional linkage. The two-dimensional linkages can include a 6-bar linkage. In some embodiments, the burrowing robot heads of the present disclosure are configured to perform a two-step, penetration-expansion motion when burrowing in or through ground (e.g., soil). The two-step, penetration-expansion process better ensures that the energy required to create a cavity in soil or other material is largely deformation energy and the frictional component of the energy is reduced significantly.

### BRIEF DESCRIPTION OF THE DRAWINGS

FIG. 1 schematically illustrates a burrowing robot in accordance with principles of the present disclosure;

FIG. 2 illustrates a two-step, penetration-expansion process or method performed by burrowing robot heads in accordance with principles of the present disclosure;

FIG. 3A is a perspective view of portions of a burrowing robot head in accordance with principles of the present disclosure and in a retracted state;

FIG. 3B is a perspective view of the burrowing robot head of FIG. 3A in an expanded state;

FIG. 4 is a simplified diagram of a portions of a burrowing robot head in accordance with principles of the present disclosure;

FIG. 5A is a simplified representation of a burrowing robot head in accordance with principles of the present disclosure and in a retracted state;

FIG. 5B is a simplified view of the burrowing robot head of FIG. 5A in an expanded state;

FIG. 6A is a perspective view of a burrowing robot head in accordance with principles of the present disclosure and in a retracted state; and

FIG. 6B is a perspective view of the burrowing robot head of FIG. 6A in an expanded state.

### DETAILED DESCRIPTION

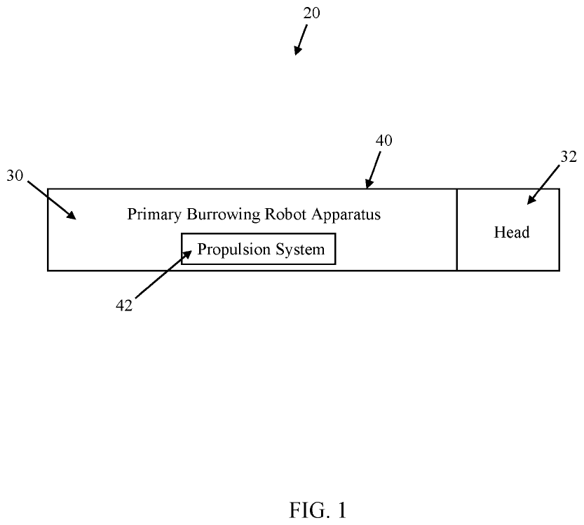

Aspects of the present disclosure relate to underground burrowing robots and heads for underground burrowing robots. One embodiment of an underground burrowing robot 20 is schematically shown in FIG. 1. The underground burrowing robot 20 generally includes a primary burrowing robot apparatus 30 and a head 32 in accordance with principles of the present disclosure. The head 32 can be used or useful with a wide variety of different burrowing robot constructions, such that the primary burrowing robot apparatus 30 can assume a plethora of differ forms or formats as currently known or in the future developed. As will be apparent to one of ordinary skill, the primary burrowing robot apparatus 30 generally includes a robot body 40 defining an exterior footprint and a propulsion system 42; the propulsion system 42 operates to manipulate or move the robot body 40 in a manner conducive to burrowing and traversing (including steering) underground. By way of non-limiting examples, the primary burrowing robot apparatus 30 can be, or can be akin to, a soft robot, such as any of the soft robots described in Kowalewski et al., U.S. Pat. No. 10,760,597 (entitled Soft Robots, Soft Actuators, and Methods for Making the Same), the entire teachings of which are incorporated herein by reference. Other non-limiting examples of borrowing-type robots useful as the primary burrowing robot system 30 include those described in Huang et al., “Modeling Clam-inspired Burrowing in Dry Sand using Cavity Expansion Theory and DEM,” Acta Geotech., vol. 15, no. 8, pp. 2305-2326, 2020; Martinez et al., “Evaluation of Self-Penetration Potential of a Bio-Inspired Site Characterization Probe by Cavity Expansion Analysis,” Can. Geotech. J., pp. 1-41, 2019; Naclerio et al., “Controlling subterranean forces enables a fast, steerable, burrowing soft robot,” Sci. Robot., 2021; Sadeghi et al., “Toward Self-Growing Soft Robots Inspired by Plant Roots and Based on Additive Manufacturing Technologies,” vol. 4, no. 3, pp. 211-223, 2017; “The Autonomous Dig: GE's Giant Earthworm Tunneling Robot Finds its Own Way,” GE Press Release, 2021. [Online]. Available: https://www.ge.com/news/press-releases/the autonomous-dig-ges-giant-earthworm-tunneling-robot-finds-its-own-way; the entire teachings of each of which are incorporated herein by reference.

Regardless of an exact design or configuration of the primary burrowing robot apparatus 30, the head 32 is configured to facilitate a burrowing movement through soil or other materials (e.g., underground materials). In this regard, the head 32 is adapted for assembly to, or to be carried by, a leading end of the robot body 40, with the primary burrowing robot apparatus 30 configured to prompt operation of, or facilitate the prompting of operation of, the head 32 in the manners as described in greater detail below.

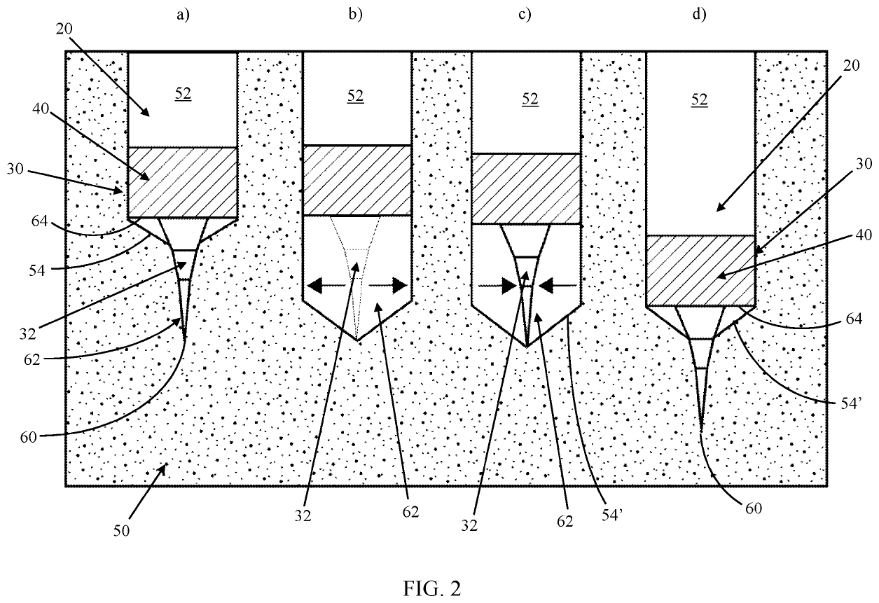

As a point of reference, movement of the burrowing robot 20 into and through ground (e.g., soil) generally entails the head 32 penetrating the ground and creating a cavity; the borrowing robot 20 can then be readily advanced into the so-created cavity via operation of the primary burrowing robot apparatus 30 (e.g., a size of the so-created cavity can accommodate the footprint of the robot body 40). With this in mind, friction is a major component of the indentation energy required by a burrowing robot's head 32 to create a cavity in cohesive soils. The burrowing robot heads of the present disclosure are configured to minimize this frictional component of the head indentation energy by performing or using a 2-step, penetration-expansion process or motion as shown, for example, in FIG. 2.

FIG. 2 represents, in simplified form, operation of the burrowing robot 20 when burrowing through ground 50. To accomplish this movement, the head 32 sequentially transitions through steps a)-d), creating a cavity along or through which the burrowing robot 20 progresses. At the stage or point in time of step a), a tunnel 52 has been formed into the ground 50 (e.g., via the operational steps described below) and the burrowing robot 20 has advanced or progressed along the tunnel 52 to the location shown. The tunnel 52 can be described as extending to or terminating at a leading surface 54 within the ground 50. The head 32 is in a retracted (or unexpanded) state. The retracted state of the head 32 defines a slender profile terminating at a pointed tip or tip-like end 60, and generally has a footprint or outer dimensions that is less than that of the robot body 40. Immediately prior to the point in time of step a), the primary burrowing robot apparatus 30 has been operated to move the burrowing robot 20 forward (i.e., downward relative to the orientation of FIG. 2), with the tip 60 readily penetrating through the surface 54 and indenting into the ground 50, creating a cavity 62 (i.e., in the state of step a), the cavity 62 corresponds with the profile of the head 32 in the retracted state). Forward movement of the burrowing robot 20 continued until a leading end 64 of the robot body 40 came into contact with the surface 54 (at which point, the ground 50 may resist further forward movement).

The head 32 is then operated to transition to an expanded state as shown at step b) (represented by arrows), thereby enlarging the cavity 62. Subsequently, the head 32 is operated to transition back to or toward the retracted state at step c) (represented by arrows at step c)), with portions of the cavity 62 no longer occupied by the head 32 now being open. In some embodiments, a footprint of the head 32 in the expanded state, and thus a size of the cavity 62, corresponds with (e.g., approximates or is greater than) a footprint or exterior size of the robot body 40. Regardless, following retraction or contraction of the head 32 (i.e., the stage of step c)), the cavity 62 serves as a newly-created extension of the tunnel 52 to a newly-created leading surface 54′. The primary burrowing robot apparatus 30 is then operated to move the burrowing robot 20 forward, with the tip 60 readily penetrating through the surface 54′ and again indenting or submerging into the ground 50 as in step d). Forward movement of the burrowing robot 20 can continue until the leading end 64 of the robot body 40 comes into contact with the surface 54′ (at which point, the ground 50 may resist further forward movement).

The steps of FIG. 2 can be repeated, with the robot body 40 readily passing into a newly-formed cavity 62 and the head 32 (in the retracted stated) readily penetrating and progressing further into the ground 50. The motion keeps repeating as the burrowing robot 20 keeps traveling downward (relative to the orientation of FIG. 2). It will be understood that these same techniques can be used to travel in any direction (and is not limited to vertically downward motion of the burrowing robot 20). Furthermore, the head 32 can assume a curved shape to aid in changing directions during the penetration process. From these explanations, the heads of the present disclosure can be considered or described as “morphing” between the retracted and expanded states.

The 2-step penetration-expansion process ensures that the energy required to create a cavity in soil or other material is largely deformation energy and the frictional component of the energy is reduced significantly. Some advantages of the burrowing robot heads and methods of the present disclosure include: 1) Lower soil penetration force compared to fixed cone heads due to initial indentation using a slender head; 2) Less energy needed to create a cavity for trailing robot body; 3) Smaller robot size since a lower balancing force needs to be provided due to reduced penetration force; 4) Higher maneuverability due to reduced size of the robot.

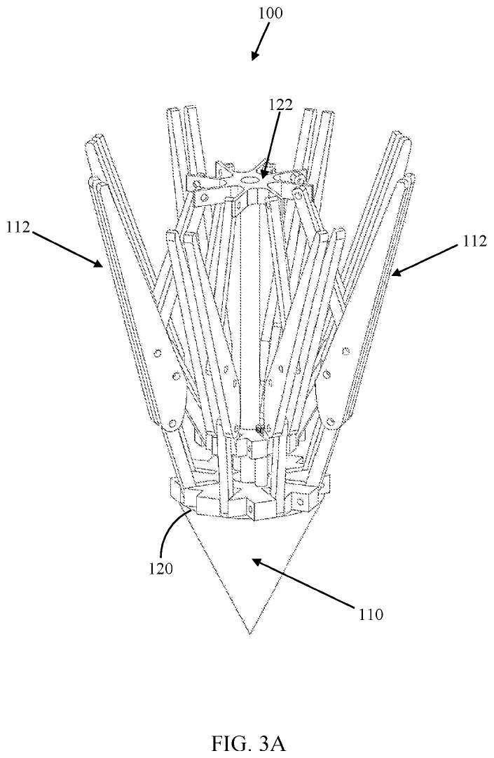

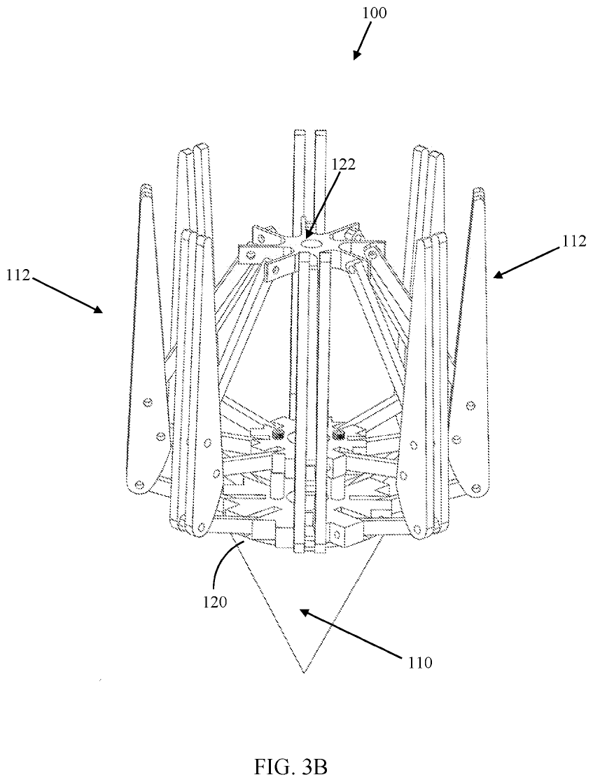

One example of a head 100 useful as the burrowing robot heads of the present disclosure is shown in FIGS. 3A and 3B. As a point of reference, the views of FIGS. 3A and 3B reflect a “skeleton” of the head 100; further, the head 100 is depicted in a retracted (or unexpanded) state in FIG. 3A, whereas FIG. 3B reflects an expanded state. The head 100 includes a tip or nose 110 and a plurality of expansion devices 112 (in some non-limiting examples, six of the expansion devices 112). In some embodiments, the expansion devices 112 can be identical and are equidistantly spaced from one another about a perimeter defined at a trailing side 120 of the tip 110. In some non-limiting examples, the tip 110 has a conical shape (e.g., a continuous cone, a shape formed by combining frustums of different cones as in FIG. 2, etc.), with the trailing side 120 thus defining a circumference about with the expansion devices 112 are arranged. In some embodiments, each of the expansion devices 112 is formed or provided as a two dimensional (2D) linkage. With the non-limiting example of FIGS. 3A and 3B, each of the 2D linkages 112 is a 6-bar linkage with one ground pivot located at a circumference of the trailing side 120 of a fixed cone of the tip 110. The expansion devices/linkages 112 have a common or connected input end 122 that is slidably associated with the tip 110. A soft and/or stretchable skin (not shown), such as a latex or natural rubber material, or other cover format is wrapped or formed around an exterior of the expansion devices 112 to create a seamless penetration head that avoids entry of soil inside the head 100. The expansion devices/linkages 112 readily transition between the retracted state (FIG. 3A) and the expanded state (FIG. 3B). In this regard, one or more actuators (not shown) can be connected to the input end 122 of the expansion devices/linkages 112. The actuator(s) can be operated upon by the primary burrowing robot apparatus 30 (FIG. 1), causing the expansion devices/linkages 112 to collectively translate between the retracted and expanded states.

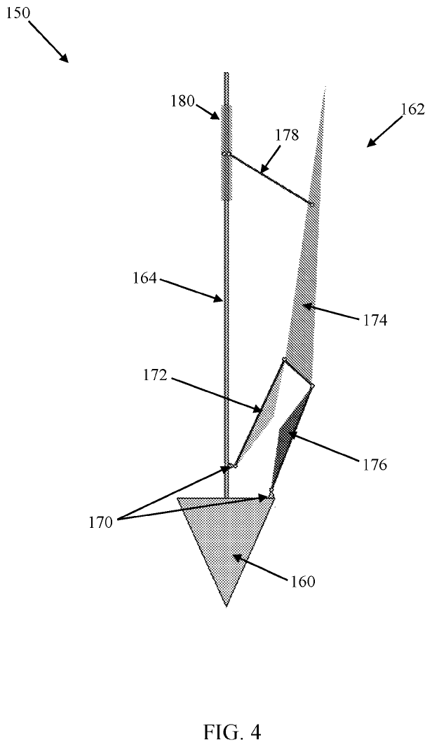

The 6-bar linkage (e.g., slider pushing on a four bar linkage) described above is but one acceptable format for the expansion devices 112. Other linkage configurations are also acceptable. In more general terms, the heads of the present disclosure can include multiple 2D linkages arranged about a perimeter of a tip or nose (e.g., a conical tip in some non-limiting examples). With this in mind, FIG. 4 illustrates portions of another head 150 of the present disclosure, including a tip or nose cone 160 and an expansion member 162 (it being understood that a plurality of the expansion members 162 will be included). A central shaft 164 is connected to and extends from the tip 160, with the tip 160 and the shaft 164 combining to define a frame for the expansion members 162. The expansion member 162 includes a 4-bar linkage comprised of a first link 170 (provided by the frame (i.e., the tip 160 and/or the shaft 164), a second link 172, a third link 174, and a fourth link 176. The 4-bar linkage is powered by a fifth link 178 and a slider 180 (that also serves as a sixth link). As the slider 180 pushes down along the shaft 164, the expansion member 162 expands radially outwardly as the third link 174 and the fourth link 176 change their position. In other embodiments, the slider 180 can be placed closer to the tip 160 and made to slide up and cause expansion. Other, similar configurations can alternatively be employed. In yet other embodiments, a completely linkage-free construction can be employed.

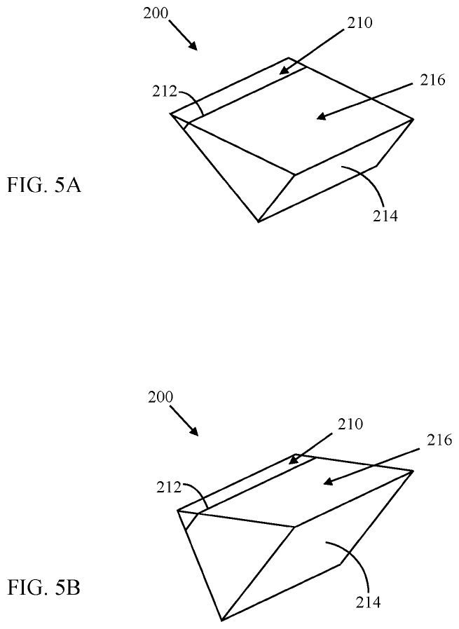

In some embodiments, heads of the present disclosure can have a conical shape (e.g., the heads 100, 150 that expand and retract between a narrow cone shape and a wider or larger diameter cone shape). In other embodiments, burrowing robot heads of the present disclosure can be wedge-shaped. For example, FIGS. 5A and 5B illustrate, in simplified form, portions of another embodiment burrowing robot head 200 of the present disclosure. As a point of reference, the head 200 is depicted in a retracted (or unexpanded) state in FIG. 5A, whereas FIG. 5B reflects an expanded state. The head 200 has a wedge-like shape and includes a tip 210. The tip 210 is configured for penetrating into ground. A plurality of expansion devices (hidden), for example linkage-type assemblies as described above, are arranged about a perimeter (e.g., parallelogram shape) of the tip 210 at a trailing side 212 thereof, and extend to a base region 214 of the head 200. The expansion members are contained within a cover 216 of the head 200, and are operable (e.g., via one or more actuators (not shown) operated upon by the primary burrowing robot apparatus 30 (FIG. 1)), to collectively move between the retracted and expanded states.

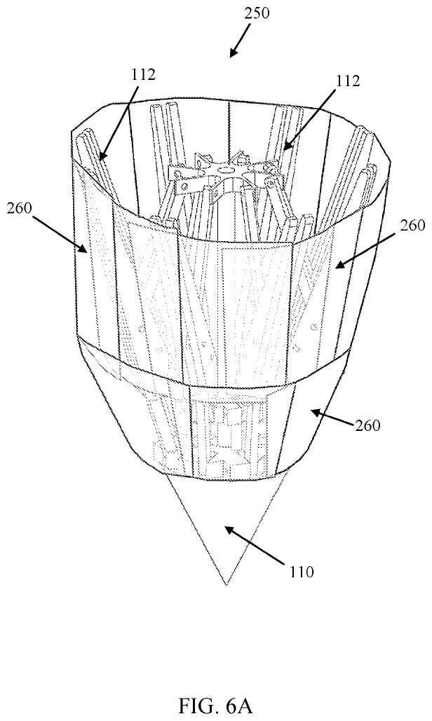

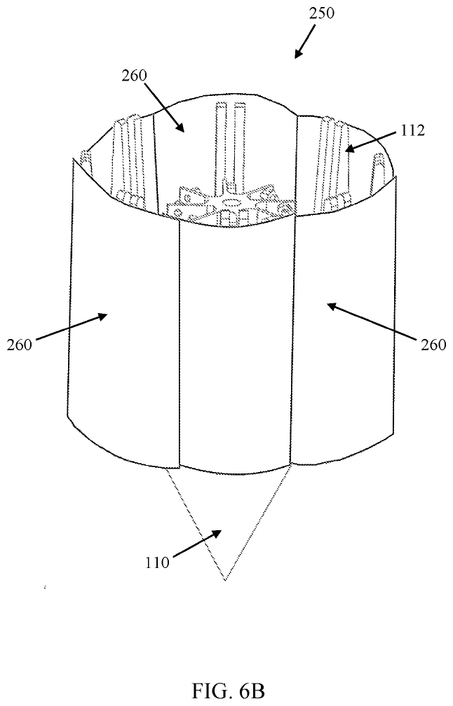

As mentioned above, in some embodiments the burrowing robot heads of the present disclosure can optionally include an outer, stretchable skin that is formed or wrapped about the expansion members. With embodiments in which the expansion members are planar linkage mechanisms radially arranged about the robot head, as the planar linkage mechanisms expand, the leave gaps between them. With these and related embodiments, the optional stretchable skin that goes around theses linkages can prevent soil from entering the head through the gaps. Other cover formats are also acceptable. For example, FIGS. 6A and 6B illustrate another burrowing robot head 250 of the present disclosure. As a point of reference, the head 250 is depicted in a retracted (or unexpanded) state in FIG. 6A, whereas FIG. 6B reflects an expanded state. The head 250 can be akin to the head 100 (FIGS. 3A and 3B) described above, and includes the tip or nose cone 110 and the plurality of expansion devices 112. In addition, the head 250 includes a plurality of flexible shields or plates 260. For ease of understanding, the shields 260 are shown as being transparent in FIG. 6A. Respective ones of the shields 260 are connected to a corresponding one of the expansion devises 112. The shields 260 are sized and shaped such that in the retracted state of FIG. 6A, adjacent ones of the shields 260 overlap one another. As the expansion devices 112 expand to or toward the expanded state of FIG. 6B, adjacent ones of the shields 260 slide along one another, and maintain a continuous surface around the head 250. A soft and/or stretchable skin (not shown) can optionally be wrapped or formed around an exterior of the shields 260. With these and related embodiments, the shields 260 support the skin and prevent the skin from being pushed inwards between the expansion devices 112. Regardless, the overlapping shields or plates 260 can be utilized with any of the burrowing robot heads of the present disclosure, and are not limited to use with the 6-bar linkage mechanism configurations of FIGS. 6A and 6B.

Although the present disclosure has been described with reference to preferred embodiments, workers skilled in the art will recognize that changes can be made in form and detail without departing from the spirit and scope of the present disclosure.

1. A burrowing robot head comprising:
a tip having a trailing end defining a perimeter; and
a plurality of expansion members arranged about the perimeter, wherein the expansion members are each configured to transition between a retracted state and an expanded state.

2. The burrowing robot head of claim 1, wherein an outer footprint of the head in the expanded state is greater than an outer footprint in the retracted state.

3. The burrowing robot head of claim 1, wherein each of the expansion members includes a linkage assembly.

4. The burrowing robot head of claim 3, wherein the linkage assembly is a two-dimensional linkage.

5. The burrowing robot head of claim 3, wherein the linkage assembly is a 6-bar linkage.

6. The burrowing robot head of claim 1, wherein the burrowing robot head is configured to perform a 2-step penetration-expansion motion.

7. The burrowing robot head of claim 1, wherein the tip has one of a conical shape and a shape formed by combining frustums of different cones.

8. The burrowing robot head of claim 7, wherein the expansion members are arranged about a circumference defined at the trailing end.

9. The burrowing robot of claim 8, wherein the expansion members collectively define an outer diameter, and further wherein a collective diameter defined by the expansion members in the retracted state is less than a collective diameter defined by the expansion members in the retracted state.

10. The burrowing robot head of claim 1, wherein the tip has a wedge shape.

11. The borrowing robot head of claim 1, further comprising:
a cover extending over an exterior of the expansion members.

12. The borrowing robot head of claim 11, wherein the cover includes a stretchable skin.

13. The borrowing robot head of claim 11, wherein the cover includes a plurality of shields, and further wherein respective ones the shields are connected to a corresponding one of the expansion members.

14. A burrowing robot comprising:
a primary burrowing robot apparatus including a robot body and a propulsion system; and
a head connecting to and extending from a leading end of the robot body, the head including:
a tip having a trailing end defining a perimeter, and
a plurality of expansion members arranged about the perimeter, wherein the expansion members are each configured to transition between a retracted state and an expanded state.

15. The burrowing robot of claim 14, wherein each of the expansion members includes a linkage assembly.

16. The burrowing robot of claim 15, wherein the linkage assembly is a two-dimensional linkage.

17. The borrowing robot of claim 14, wherein the head is configured to perform a 2-step penetration-expansion motion.

18. The burrowing robot of claim 14, wherein the head further includes a cover extending over an exterior of the expansion members.

19. A method for advancing a burrowing robot through ground, the burrowing robot including a primary burrowing robot apparatus and a head, the primary burrowing robot apparatus include a robot body and a propulsion system, wherein the head extends from a leading end of the robot body, the method comprising:
arranging the head in a retracted state;
penetrating a surface of the ground with a tip end of the head, with the head in the retracted state, such that the head indents into the ground;
expanding the head to an expanded state, wherein the step of expanding includes the head displacing a region of the ground;
retracting the head back to the retracted state;
wherein following the step of retracting, a cavity is defined in the ground; and
advancing the robot body into the cavity.

20. The method of claim 19, wherein the head has one of a conical shape, a shape formed by combining frustums of different cones, and a wedge shape.
